(19) United States
(12) Patent Application Publication
(10) Pub. No.: US 2009/0325314 A1
(43) Pub. Date: Dec. 31, 2009

(54) N-Methyl Scanning Mutagenesis

(75) Inventors: Stephen V. Fiacco, Beverly Hills, CA (US); Richard W. Roberts, South Pasadena, CA (US)

Correspondence Address: HOGAN & HARTSON L.L.P., 1999 AVENUE OF THE STARS, SUITE 1400, LOS ANGELES, CA, 90067, US

(73) Assignee: UNIVERSITY OF SOUTHERN CALIFORNIA, Los Angeles, CA (US)

(21) Appl. No.: 12/479,331

(22) Filed: Jun. 5, 2009

Related U.S. Application Data
(60) Provisional application No. 61/059,202, filed on Jun. 5, 2008.

(51) Int. Cl. G01N 33/566; G01N 33/00; C07K 14/00; C07K 7/08

(52) U.S. Cl. 436/501; 436/86; 530/300; 530/326

(57) ABSTRACT

The present invention relates to methods and compositions comprising the insertion of a single N-methyl amino acid into functional peptides. More specifically, the invention discloses methods referred to as N-methyl scanning mutagenesis, where one or more N-methyl amino acid substitutions into functional peptides enhances protease resistance while retaining binding affinity.

The present application claims the benefit of the filing date of U.S. Provisional Application No. 61/059,202 filed Jun. 5, 2008, the disclosure of which is incorporated herein by reference in its entirety.

### STATEMENT REGARDING FEDERALLY SPONSORED RESEARCH OR DEVELOPMENT

This invention was made with government support under Contract No. R01 60416 and R21 76678 awarded by the National Institutes of Health. The government has certain rights in the invention.

### FIELD OF THE INVENTION

The present invention relates in general to N-methyl scanning mutagenesis. More specifically, the invention provides methods and compositions comprising N-methyl peptides with enhanced stability and function.

### BACKGROUND OF THE INVENTION

Peptide ligands often suffer from poor bioavailability due in part to proteolytic degradation in vivo. Previous work has shown that homo-oligomers of N-methyl Phe are highly resistant to proteolysis from either chymotrypsin or proteinase K [1]. This work also showed that oligopeptides made with a mixture of Phe and N-methyl Phe retain some protease resistance. Similarly, peptoids, polymers of N-alkyl glycine, show resistance to proteolysis [2] and may enhance affinity and specificity when substituted for proline [3]. Unfortunately, movement of the α-carbon sidechain to the amide nitrogen often disrupts peptide function.

### SUMMARY OF THE INVENTION

In one embodiment, the invention relates to methods for enhancing stability and preserving function of peptides The method comprises inserting single N-methyl containing amino acids into peptides and comparing the stability and function of the N-methyl peptide with the stability and function of a non-N-methylated control peptide. If the stability of the N-methyl peptide is greater than the stability of a control peptide, then the stability has been enhanced. If the N-methyl peptide provides the same function as compared to the control peptide, then the function of the peptide has been preserved.

In a related embodiment, the invention relates to methods for enhancing stability and function of peptides. The method comprises inserting single N-methyl amino acids into a peptide and comparing the stability and function of the N-methyl peptide with the stability and function of a control peptide. If the stability of the N-methyl peptide is greater than the stability of a control peptide and the function of the N-methyl peptide is greater than the function of a control peptide, then the stability and function of the peptide has been enhanced.

Within one aspect, the present invention provides peptides comprising a single N-methyl amino acid that has enhanced stability and function as compared to a control peptide.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

FIG. 1. Peptide stability to proteolysis. (A) Site of trypsin digestion. (B) Proteolysis of unmodified peptide (▾), N-Me-L3 (▪), N-Me-K (), N-Me-Y (♦), and N-Me-D (▴) by trypsin.

FIG. 2. Relative Peptide Affinities. (A) Radiolabeled Gαi1 was incubated with immobilized, biotinylated R6A and varying concentrations of either R6A (▾), unmodified peptide (▴), or N-Me-K (♦) for 3 hr. at 4° C. The radiolabel bound was normalized to that which bound in the absence of competitor. The plotted values represent the mean of three experimental values and the error bars correspond to the standard deviation. (B) Experimentally determined binding data is shown above. The error is calculated based on the standard error of the mean (SEM) for the log IC50 using GraphPad 5.0.

FIG. 3. Binding of radiolabeled Gα subunits to immobilized peptides. Gαi1, Gαi2, Gαi3, Gq, Gs, GoαA, Gα12, and Gα15 pcDNA were translated into 35S-Met labeled proteins using TnT coupled transcription/translation system (Promega). Translation reactions were desalted and applied to (A) Unmodified peptide, (B) N-Me-K, and (C) N-Me-L3 immobilized to Neutravidin-agarose. Values represent the mean value from three experiments and the error bars represent the standard deviation.

### DETAILED DESCRIPTION OF THE INVENTION

The inventors were interested in strategies that enhance proteolytic stability while preserving the function of the underlying peptide. Toward that end, the inventors have explored the effect of inserting single N-methyl amino acids into functional peptides, a method referred to as N-methyl scanning mutagenesis. The inventors hope was to find one or more substitutions that enhance protease resistance while retaining binding affinity.

The inventors chose the G protein binding core motif peptide DKLYWWEFL, which binds Gαi1*GDP with good affinity (Kd=200 nM), as our model system [4]. The strategy was to systematically construct 9 variants of this peptide bearing a single N-methyl analogue of the natural residue at each position. The challenge faced was that previous synthetic methods for incorporating N-methyl residues involved long multi-step reactions per coupling or resulted in low yields [5,6]. The synthetic difficulty is the result of coupling the secondary amine of an N-methyl peptide [7]. This coupling must go to near completion, in a short period of time to avoid diketopiperazine formation [6]. To couple N-methyl residues the inventors followed a strategy similar to Giralt and coworkers [6], but used the additive pair HATU/HOAT. Coupling each N-methyl amino acid goes to near completion in 20 minutes in one step with no pre-activation for all residues we tested. As a result, the inventors were able to construct the N-methyl peptides reliably with a final yield of 20-25% (Table 1).

TABLE 1
N-methyl peptides examined

| Compound | Sequence |
|---|---|
| N-Me-D | NNNNN(N-MeD)KLYWWEFL |
| N-Me-K | NNNNND(N-MeK)LYWWEFL |
| N-Me-L3 | NNNNNDK(N-MeL)YWWEFL |
| N-Me-Y | NNNNNDKL(N-MeY)WWEFL |
| N-Me-W10 | NNNNNDKLY(N-MeW)WEFL |
| N-Me-W11 | NNNNNDKLYW(N-MeW)EFL |
| N-Me-E | NNNNNDKLYWW(N-MeE)FL |
| N-Me-F | NNNNNDKLYWWE(N-MeF)L |
| N-Me-L14 | NNNNNDKLYWWEF(N-MeL) |

Peptides were synthesized with and without biotin conjugation at the N-terminus. Additionally five N-terminal Asn residues were added to enhance peptide solubility.

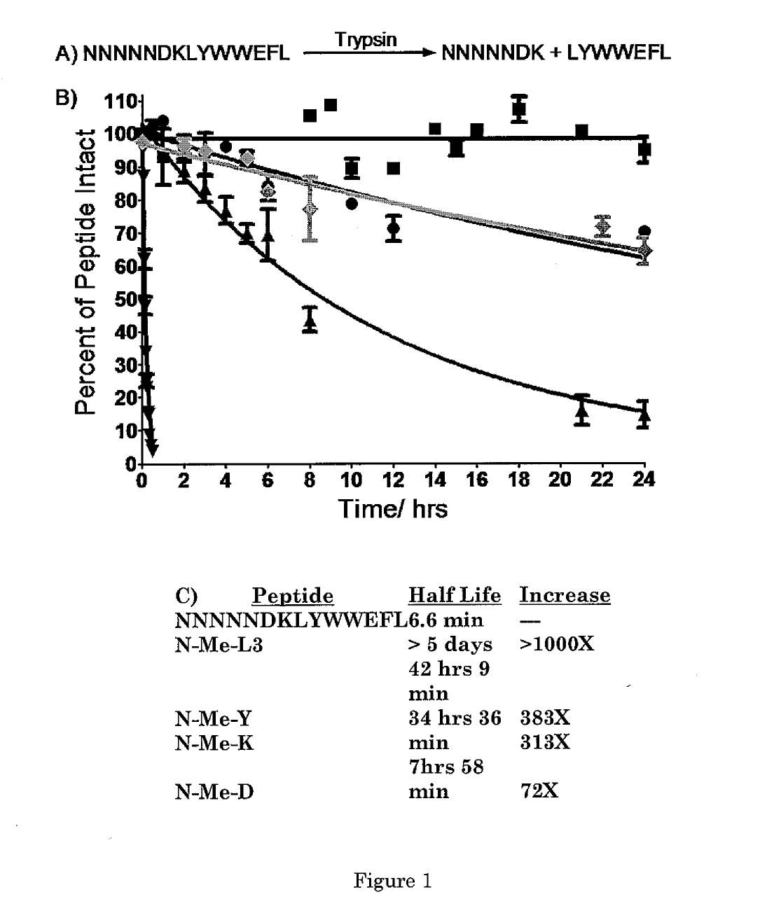

To investigate the effect of N-methylation on protease activity, the half-life was measured for trypsin cleavage for N-methyl substitutions at the P2, P1, P1′, and P2′ positions (N-Me-D, N-Me-K, N-Me-L3, and N-Me-Y respectively) (FIG. 1). Inserting N-methyl residues at these positions results in a dramatic increase in proteolysis resistance, ranging from 72 to >1000-fold (FIG. 1C). Surprisingly, the site of N-methyl incorporation does not need to be at scissile bond or at the trypsin recognition site in order to confer this stability. The observations indicate that inserting a single N-methyl residue substantially reduces proteolysis in a 4-residue window.

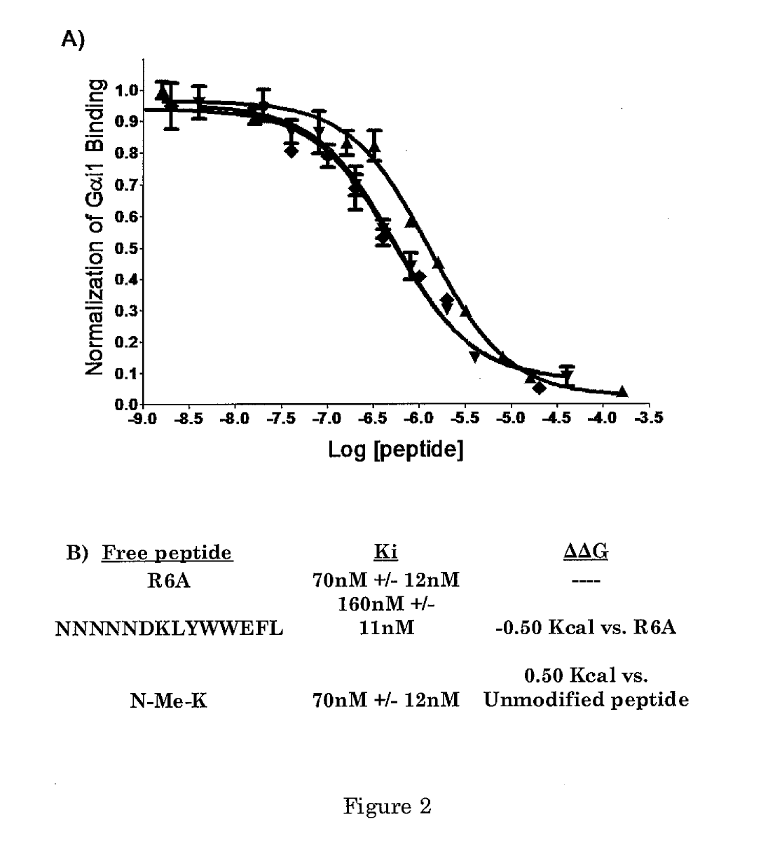

The inventors next worked to determine if N-methyl insertions altered the peptide binding affinity to Gαi1·GDP. While N-methyl amino acid incorporation generally reduced binding, they observed increased binding affinity for N-Me-K. The inventors quantitated this increase using via equilibrium competition using the R6A peptide (MSQTKRLDDQLYWWEYL; Kd=60 nM)[8]. This analysis indicated that N-Me-K enhanced binding by 2.5-fold, a ΔΔG of −0.50 kcal/mol (FIG. 2 A, B).

The crystal structure of R6A in complex with Gαi1·GDP (S. Sprang, A. Adhikari, personal communication) provides insight into the binding data. The N-methyl substitutions would disrupt many of the peptide-protein interactions or the fold of the bound peptide. From the crystal structure of a similar sequence a hydrogen bond is present from the backbone of V (equivalent to L in our peptide) to Gαi1·GDP is observed [9]. This would indicate that N-methyl incorporation at this position would disrupt binding to Gαi1·GDP, which the inventors observed. Additional insight may be inferred by the results of other peptides selected by mRNA display to bind to Gαi1·GDP, where a significant conservation of the EFL residues have been observed [10]. Many mutations are subtle, E to D or F to Y for example. Therefore it would be suspected that N-methyl incorporation at any of these positions would be deleterious to peptide binding. This was experimentally confirmed, as the inventors peptide binding to Gαi1·GDP.

The Q residue in R6A (equivalent to K in our peptide) adopts Phi, Psi angles of −60, 128 respectively. Previous work indicated that peptoids have a limited number of energetically favorable conformations [11]. It is likely that the incorporation of an N-methyl amino acids results in a conformation similar to an optimal orientation for Gαi1 binding. This leads the inventors to a conformational hypothesis for N-methylation. Assuming no unfavorable steric interactions, the inventors predicted that N-methyl amino acid incorporation would have been favorable or neutral when Phi, Psi angles are similar to those found in peptoids [11]. This heuristic should provide a good starting point for N-methyl amino acid modifications that improve peptide stability without impairing function. Further structural analysis may be beneficial and will be carried out in future work.

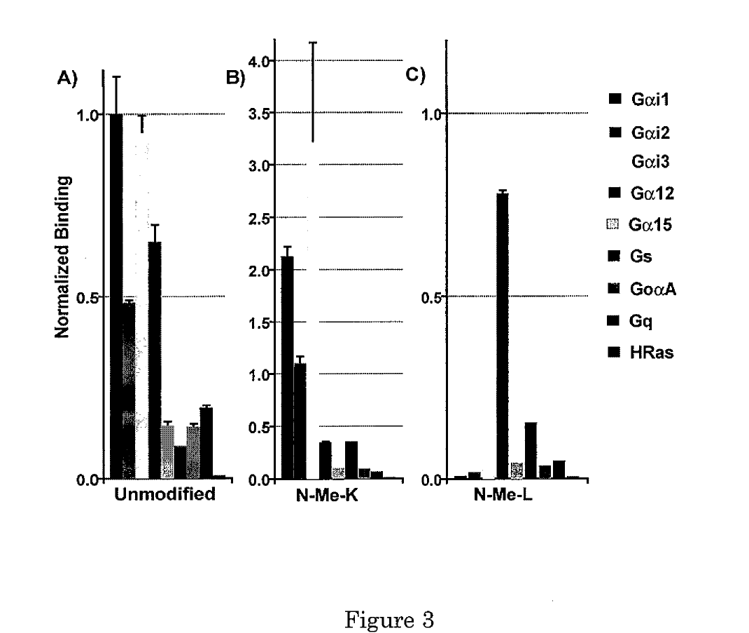

The inventors wanted to see if N-methyl incorporation altered the specificity of the peptides. Members of the peptide library were tested for binding against 9 members of the G-protein family known to have various affinities to R6A. 5 of the 9 N-methyl peptide variants showed a dramatic increase in specificity. The variant showed specific affinity for Gα12 over Gαi1 which the original selection targeted (FIG. 3). This results in the first peptide ligand able to specifically bind to Gα12.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

Experimental Section

Peptide Synthesis: All solvents were purchased from Sigma. All peptides except N-Me-L14 were synthesized by manual solid-phase peptide synthesis with (250 mgs, 0.15 mmoles) preloaded Leu-2-Chlorotrityl resin (Anaspec). N-Me-L14 was synthesized after loading 2-Cholortrityl resin (Anaspec) with Fmoc-N-methyl Leucine (Anaspec). 5 N-terminal Asn residues were added to enhance peptide solubility. Loading was accomplished by adding 5 eq. of Fmoc-N-Me-L14 to (250 mgs, 0.35 mmoles) 2-Cholortrityl resin in DMF with 5 eq. DIEA for 2 hrs. Standard couplings were carried out with 5 eq. of monomer (Novabiochem) in (2 mL, 0.6 mMoles) HATU (Novabiochem), (1.2 mMoles) HOAt (Genescript) in DMF with (1.8 mMoles) DIEA at room temperature for 15 min. Coupling to an N-methyl amino acid followed the same procedure with a 30 min coupling time. Fmoc deprotection was carried out with 20% piperidine (Anaspec) at room temperature for 15 min. Following, deprotection, cleavage with 95% TFA, filtration and ether extraction, the crude product was purified on a Vydac C-18 reverse phase column using gradient elution (0% B for 5 min, 10-50% B in 40 min. Solvent A: H2O with 0.1% TFA, Solvent B: CH3CN with 0.035% TFA. Lyophilized solid was reconstituted in DMSO and quantitated by absorbance at 280 nm (ε₂₈₀=12490 L mol⁻¹ cm⁻¹). Yield=20-25%.

Peptides were characterized by HPLC and MALDI-TOF. Peptides were run on a Vydac C-18 column using gradient elution (5% B to 95% B). Buffers were the same as described above. Absorbance at 280 was recorded for biotin labeled peptides excluding Bio-N-Me-D, Bio-R6A and Bio-unmodified peptide. Absorbance at 215 nm were recorded for the remaining peptides. The peak around 4 min for peptides at 215 nm was the solvent peak. The next major peak (or first major peak in the case of 280 nm recording) was collected and analyzed by MALDI-TOF.

Binding affinity of N-methyl peptide library: TnT pulldown experiments were carried out as described in [10] with some modifications. Human cDNA clones encoding G proteins with the pcDNA3.1+ vector (Invitrogen) were obtained from the UMR cDNA Resource Center (www.cdna.org). The DNA clones for Gαi1, and HRas were translated into 35S-Met labeled proteins using a TnT coupled transcription/translation system (Promega). Translation reactions were desalted using G-25 spin columns (GE Healthcare) and the translation yield was calculated by TCA precipitation. Equal amounts of each labeled subunit were added to (5 μL) neutravidin-agarose containing (2 nmol) pre-bound biotin labeled peptide in (1 mL) HBS-EP buffer. Control neutravidin-agarose beads were loaded with (d)-biotin (Anaspec). Binding reactions proceeded for 2 hrs at 4° C. followed by filtration and washing with HBS-EP. The matrix was analyzed by scintillation counting and the percent bound was determined by the matrix counts divided by the total counts as determined by TCA precipitation. Values were normalized to the percent bound of unmodified peptide.

R6A-1 Competition/Equilibrium Binding: R6A (MSQTKRLDDQLYWWEYL) and Bio-R6A (Bio-MSQTKRLDDQLYWWEYL) were synthesized by previously described methods. This peptide was immobilized to NeutrAvidin beads (Promega) using the manufacturer's instructions. Gαi1 was expressed using the TnT reticulocyte lysate system (T7 promoter, Promega). (250 ng) plasmid DNA and (25 μCi) L-35S-Met (MP Biomedicals) were used in an (25 μL) expression. Gαi1 translations were desalted using MicroSpin G-25 columns (GE healthcare) into modified HBS-EP buffer [10 mM HEPES at pH 7.4, 150 mM NaCl, 3 mM EDTA, 0.005% polysorbate 20 (Tween 20), 8 mM MgCl2, 30 μM GDP, and 0.05% (w/v) BSA]. Specific Activity was measured by TCA precipitation.

400 pmol immobilized R6A, and (100,000 cpm) Gαi1 were suspended in (1 mL) HBS-EP buffer. Varying quantities of free peptide (R6A, Unmodified peptide, or N-Me-K) were added. The final DMSO concentration was adjusted to 0.5%. Samples were rotated for 3 hrs at 4° C. followed by brief centrifugation and supernatant removal. Samples were washed four time by resuspension in modified HBS-EP buffer at 4° C., centrifugation, and supernatant removal. After the final wash, the immobilized sample was transferred to scintillation vials and analyzed by scintillation counting. CPM values were normalized by dividing by the average value obtained in the absence of added peptide. Binding analysis was performed by GraphPad Prism 4.0.

N-Methyl Selectivity: Gαi1, Gαi2, Gαi3, Gq, Gs, GoαA, Gα12, and Gα15 pcDNA were translated into 35^{S}-Met labeled proteins using TnT coupled transcription/translation system (Promega). Translation reactions were desalted using G-25 spin columns (GE Healthcare) and applied to peptide immobilized to neutravidin-agarose as previously described. The immobilized peptide was N-Me-D, N-Me-K, N-Me-L3, N-Me-Y, N-Me-W10, N-Me-W11, N-Me-E, N-Me-F, or N-Me-L14 as illustrated. Binding was allowed to proceed for 3 hrs at 4° C. after which the resin was filtered and washed. Scintillation counting of the resin yielded the cpm bound which was normalized as detailed in the supplemental section. Values represent the mean value from three experiments and the error bars represent the standard deviation. Binding analysis and normalization performed as outlined in R6A-1 competition/equilibrium binding.

Protease Resistance Experiment: (2 uL) Unmodified peptide, N-Me-D, N-Me-K, and N-Me-L3 (7.5 mM) in DMSO were added to (98 μL) buffer containing 50 mM Tris, 20 mM CaCl2, pH=7.75. This was used to reconstitute (20 μg) modified trypsin (New England Biolabs). The reaction proceeded at 37° C. for either 30 minutes (unmodified peptide) or 24 hrs (N-Me-D, N-Me-K, N-Me-L3, N-Me-Y). An aliquot (8 μL) was removed for each time point and diluted in aceetonitrile (25 μl). Samples were flash frozen until analysis was to be performed. At this time 0.1% TFA in H2O (67 μL) was used to dilute the samples to 100 μL. The sample was injected onto a C-18 reverse phase column and separated by gradient elution (5-90% B in 10 min. Solvent A: H2O with 0.1% TFA, Solvent B: CH3CN with 0.035% TFA). The area under the starting material peak was quantitated using the 32 KaratGold Software package (Beckman). The plotted values represent the mean of three experimental value and the error bars represent the standard error of the mean. The graph was generated by fitting the data to a one phase exponential decay equation (GraphPad Prism).

The inventors have shown that the incorporation of N-methyl amino acids can dramatically increase the function and stability of a peptide selected by mRNA display. Selective incorporation of N-methyl amino acids into peptides with a Phi, Psi angle similar to those stated for N-Me-K would likely have the same benefits from their incorporation. This relative synthetic ease and predictability makes incorporating N-methyl amino acids into peptides derived from selections a practical first step in increasing bioavailability.

Many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

1. Frankel, A.; Millward, S. W.; Roberts, R. W. Chemistry & Biology 2003, 10, 1043-1050.
2. Miller, S. M.; Simon, R. J.; Ng, S.; Zuckermann, R. N.; Kerr, J. M.; Moos, W. H. Bioorganic & Medicinal Chemistry Letters 1994, 4, 2657-2662.
3. Nguyen, J. T.; Turck, C. W.; Cohen, F. E.; Zuckermann, R. N.; Lim, W. A. Science 1998, 282, 2088-2092.
4. Ja, W. W.; Adhikari, A.; Austin, R. J.; Sprang, S. R.; Roberts, R. W. J. Biol. Chem. 2005, 280, 32057-32060.
5. Biron, E.; Kessler, H. J. Org. Chem. 2005, 70, 5183-5189.
6. Teixido, M.; Albericio, F.; Giralt, E. Journal of Peptide Research 2005, 65, 153-166.
7. DiGioia, M. L.; Leggio, A.; Liguori, A. J. Org. Chem. 2005, 70, 3892-3897.
8. Ja, W. W.; Roberts, R. W. Biochemistry 2004, 43, 9265-9275.
9. Johnston, C. A.; Siderovski, D. P. Proceedings of the National Academy of Sciences 2007, 104, 2001-2006.
10. Millward, S. W.; Fiacco, S.; Austin, R. J.; Roberts, R. W. ACS Chem. Biol. 2007, 2, 625-634.
11. Simon, R. J.; Kania, R. S.; Zuckermann, R. N.; Huebner, V. D.; Jewell, D. A.; Banville, S.; Ng, S.; Wang, L.; Rosenberg, S.; Marlowe, C. K.; Spellmeyer, D. C.; Tan, R.; Frankel, A. D.; Santi, D. V.; Cohen, F. E.; Bartlett, P. A. PNAS 1992, 89, 9367-9371.

What is claimed is:

1. A method for enhancing stability and function of a peptide comprising:
(a) obtaining a functional sample peptide;
(b) inserting a single N-methyl amino acid into said peptide and;
(c) comparing the stability and function of said sample peptide with the stability and function of a control peptide;
wherein a determination that the stability and function of said sample peptide is greater than the stability and function of a control peptide is indicative of the enhancement of stability and function of said sample peptide.

2. The method according to claim 1, wherein said stability is determined by measuring proteolysis resistance.

3. The method according to claim 1, wherein said N-methyl amino acid is inserted at the P2, P1, P1′, or P2′ position.

4. The method according to claim 1, wherein said function is determined using equilibrium competition binding assays.

5. The method according to claim 4, wherein the binding of said sample peptide is enhanced by 2.5 fold.

6. The method according to claim 1, wherein said sample peptide comprises the G peptide binding core motif of DKLYWWEFL.

7. The method according to claim 1, wherein said sample protein comprises Phi, Psi angles similar to NNNNND(N-MeK)LYWWEFL.

8. The method according to claim 1, wherein the N-methyl amino acid is a structural analogue or mimic of the residue it replaces.

9. The method according to claim 1, wherein the N-methyl amino acid has an identical sidechain to the residue it replaces.

10. The method according to claim 1, wherein the N-methyl amino acid is inserted at a position in the peptide where the Phi and Psi angles of the residue replaced lie in the 2nd quadrant of a Ramachandran Plot and has negative Phi angles, Positive Psi angles.

11. The method according to claim 1, wherein the N-methyl amino acid is inserted at a position in the peptide where the Phi and Psi angles of the residue replaced range from Phi=0 to −90 degrees and Psi=+90 to +180 degrees.

12. The method according to claim 1, wherein the N-methyl bearing peptide has improved selectivity for its target.

13. The method according to claim 1, wherein said sample peptide has improved selectivity as compared to the control peptide for a new target that is structurally homologous to the original target.

14. The method according to claim 1, wherein said sample peptide has improved selectivity as compared to the control peptide for a new target that is sequence homologous to the original target.

15. The method according to claim 1, wherein said sample peptide has improved selectivity as compared to the control peptide for a new target that is phylogenetically related to the original target.

16. The method according to claim 1, wherein said sample peptide has improved selectivity as compared to the control peptide for a target that is >10% sequence identical to the original target.

17. The method according to claim 1, wherein said sample peptide has improved selectivity as compared to the control peptide for a target that is >20% sequence identical to the original target.

18. The method according to claim 1, wherein said sample peptide has improved selectivity as compared to the control peptide for a target that is >30% sequence identical to the original target.

19. A method for enhancing stability and preserving function of a peptide comprising:
(a) obtaining a functional sample peptide;
(b) inserting a single N-methyl amino acid into said peptide and;
(c) comparing the stability and function of said sample peptide with the stability and function of a control peptide;
wherein a determination that the stability of said sample is greater than the stability of a control peptide; and
wherein a determination that said sample peptide is functional is indicative of preservation of the function of said sample peptide.

20. The method according to claim 19, wherein said stability is determined by measuring proteolysis resistance.

21. The method according to claim 19, wherein said N-methyl amino acid is inserted at the P2, P1, P1′, or P2′ position.

22. The method according to claim 19, wherein said function is determined using equilibrium competition binding assays.

23. The method according to claim 22, wherein said sample peptide is able to bind in said binding assays.

24. The method according to claim 19, wherein said sample peptide comprises the G peptide binding core motif of DKLYWWEFL.

25. The method according to claim 19, wherein said sample protein comprises Phi, Psi angles similar to NNNNND(N-MeK)LYWWEFL.

26. The method according to claim 19, wherein the N-methyl amino acid is a structural analogue or mimic of the residue it replaces.

27. The method according to claim 19, wherein the N-methyl amino acid has an identical sidechain to the residue it replaces.

28. The method according to claim 19, wherein the N-methyl amino acid is inserted at a position in the peptide where the Phi and Psi angles of the residue replaced lie in the 2nd quadrant of a Ramachandran Plot and has negative Phi angles, Positive Psi angles.

29. The method according to claim 19, wherein the N-methyl amino acid is inserted at a position in the peptide where the Phi and Psi angles of the residue replaced range from Phi=0 to −90 degrees and Psi=+90 to +180 degrees.

30. The method according to claim 19, wherein the N-methyl bearing peptide has altered selectivity for its target.

31. The method according to claim 19, wherein said sample peptide has altered selectivity as compared to the control peptide for a new target that is structurally homologous to the original target.

32. The method according to claim 19, wherein said sample peptide has altered selectivity as compared to the control peptide for a new target that is sequence homologous to the original target.

33. The method according to claim 19, wherein said sample peptide has altered selectivity as compared to the control peptide for a new target that is phylogenetically related to the original target.

34. The method according to claim 19, wherein said sample peptide has altered selectivity as compared to the control peptide for a target that is >10% sequence identical to the original target.

35. The method according to claim 19, wherein said sample peptide has altered selectivity as compared to the control peptide for a target that is >20% sequence identical to the original target.

36. The method according to claim 19, wherein said sample peptide has altered selectivity as compared to the control peptide for a target that is >30% sequence identical to the original target.

37. A peptide comprising a single N-methyl amino acid that has enhanced stability and function as compared to a control peptide.

38. The peptide according to claim 37, wherein said peptide comprises the G peptide binding core motif of DKLYWWEFL.

39. The peptide according to claim 37, wherein said peptide comprises a N-methyl amino acid inserted at the P2, P1, P1′, or P2′ position.

40. The peptide according to claim 37, wherein said peptide comprises Phi, Psi angles similar to NNNNND(N-MeK)LYWWEFL.

41. The method according to claim 37, wherein said sample protein comprises Phi, Psi angles similar to NNNNND(N-MeK)LYWWEFL.
